(19) United States
(12) Patent Application Publication
(10) Pub. No.: US 2012/0128781 A1
(43) Pub. Date: May 24, 2012

(54) FUNCTIONALIZATION OF NANOPARTICLES BY GLUCOSAMINE DERIVATIVES

(76) Inventors: Jackie Y. Ying, Singapore (SG); Nikhil R. Jana, Singapore (SG); Nandanan Erathodiyil, Singapore (SG)

(21) Appl. No.: 12/598,410

(22) PCT Filed: May 2, 2008
(86) PCT No.: PCT/SG08/00160

§ 371 Date: Feb. 1, 2011

Related U.S. Application Data
(60) Provisional application No. 60/924,160, filed on May 2, 2007.

(51) Int. Cl. A61K 47/36; C08B 37/08; A61K 9/14; B82Y 5/00; B82Y 30/00; B82Y 40/00

(52) U.S. Cl. 424/493; 536/20; 514/777; 977/773; 977/810; 977/811; 977/774; 977/754; 977/840; 977/906; 977/927

(57) ABSTRACT

The present invention relates to oligomeric or polymeric saccharide derivatives comprising glucosamine moieties, e.g. derivatives of oligomeric or polymeric glucosamines such as chitosan oligomers or polymers, in which one or more amine groups are substituted by anchoring groups that chemisorb to the surface of a nanoparticle or form an interdigitated bilayer with a surfactant layer surrounding the nanoparticle. The invention also relates to functionalized nanoparticles comprising such derivatives, a method for forming the functionalized particles and to uses thereof as molecular imaging agents, biosensing agents or drug delivery agents, or in the preparation of such agents.

### CROSS-REFERENCE TO RELATED APPLICATIONS

This application claims the benefit of U.S. Provisional Patent application Ser. No. 60/924,160, filed May 2, 2007, entitled “FUNCTIONALIZATION OF NANOSPHERES AND NANORODS BY CHITOSAN OLIGOSACCHARIDE DERIVATIVES”, the contents of which are hereby incorporated by reference.

### FIELD OF THE INVENTION

This invention relates to derivatives suitable for functionalization of nanoparticles, such as nanospheres and nanorods, to their use in preparing functionalized nanoparticles, and to the functionalized nanoparticles obtained. The invention also relates to the use of the obtained functionalized nanoparticles as molecular imaging agents, biosensing agents or drug delivery agents, or for their use in the preparation of such molecular imaging agents, biosensing agents or drug delivery agents.

### BACKGROUND OF THE INVENTION

Nanoparticles have a wide range of applications in chemical and biomedical fields due to their unique size-dependent properties.¹ Although several methods have been developed for the size-controlled synthesis of noble metals, quantum dots and magnetic oxides, the as-prepared nanoparticles are hydrophobic in nature, and functionalization remains a challenge for their applications, especially in biological systems.²

There are two common strategies to convert hydrophobic nanoparticles into hydrophilic and functionalized nanoparticles, being ligand exchange of the original surfactant with hydrophilic ligands such as thiols³ or other functional groups,¹ and the second being the formation of an interdigitated bilayer between amphiphilic molecules/polymers and a passivating surfactant layer surrounding the nanoparticle.⁴ Although both approaches have been applied to noble metals, iron oxide and quantum dots, each approach has certain limitations, such as weak chemical interaction of ligands with the nanoparticle surface, poor stability of interdigitated bilayer, and nanoparticle growth/aggregation during ligand-exchange processes, which limitations can lead to poor colloidal stability¹. Various modifications of these strategies have been developed, e.g. use of ligands with multiple thiols, thiolated dendrimers and dendrons,^{5a-c} and crosslinking of surface ligands/polymers.^{1c,5d,e}

Functionalized gold nanoparticles, such as nanospheres and nanorods, are specifically of interest for applications in the optical detection of biomolecules. However, the colloidal stability of ligand-exchanged gold nanoparticles is usually poor, and they often precipitate during chemical modification and functionalization.^{1a,7} Gold nanorod functionalization is particularly difficult due to the associated shape change and self-assembly based aggregation during the functionalization process.⁶ Despite these limitations, some methods for gold nanorod functionalization have been reported, e.g. by ligand-exchange with thiolated molecules,⁷ by silica coating,⁸ by partial ligand-exchange with phosphatidyl choline,⁹ and layer-by-layer approach for polymer coating.¹⁰

### BRIEF SUMMARY OF THE INVENTION

In one aspect, the present invention provides a derivative of an oligomeric or polymeric saccharide comprising glucosamine moieties, in which one or more amine groups are substituted by anchoring groups that chemisorb to the surface of a nanoparticle or form an interdigitated bilayer with a surfactant layer surrounding the nanoparticle. In one embodiment the oligomeric or polymeric saccharide can be an oligo- or poly-glucosamine. In a further embodiment, the oligomeric or polymeric saccharide can be a chitosan oligomer or polymer.

In another aspect, the present invention provides a functionalized nanoparticle comprising a nanoparticle and the derivative as defined herewith.

In still another aspect, the present invention provides a method for forming a functionalized particle as defined herewith, comprising reacting a derivative of the invention with a nanoparticle.

In a further aspect, the present invention provides a use of the functionalized nanoparticle as defined herewith as a molecular imaging agent, a biosensing agent or a drug delivery agent, or in the preparation of such agents.

The above and other features and advantages of the present invention will become apparent from the following description when taken in conjunction with the accompanying figures which illustrate preferred embodiments of the present invention by way of example.

### BRIEF DESCRIPTION OF THE DRAWINGS

Embodiments of the invention will be discussed with reference to the following Figures:

FIG. 1 shows two possible coating schemes for the modification of a gold nanoparticle with thiol and oleoyl chitosan derivatives;

FIG. 2 displays UV-visible absorption spectra of gold nanoparticles (2a-nanosphere; 2b-nanorod) before (—) and after ( [symbol] ) ligand exchange;

FIG. 3 displays Transition Electron Microscope (TEM) micrographs of a chitosan derivative modified gold nanoparticles (3a-nanosphere; 3b-nanorod);

FIG. 4 displays UV-visible absorption spectra of biotinylated gold nanoparticles (4a-Au nanosphere; 4b-Au nanorod) before (—) and after ( [symbol] ) aggregation in the presence of 10 μM of streptavidin;

FIG. 5 displays a ¹H NMR (D₂O) spectra of a thiol-functionalized chitosan derivative (FIG. 5a) and of a gold nanosphere coated with the derivative (FIG. 5b);

FIG. 6 displays a ¹H NMR (DMSO-d6) of an oleic-functionalized chitosan oligomer (FIG. 6a) and of a gold nanorod coated with the oligomer (FIG. 6b).

### DETAILED DESCRIPTION OF THE INVENTION

### Glucosamine-Comprising Saccharide Derivatives

The derivative as described herein comprises an oligomeric or polymeric saccharide, which saccharide comprises a number of glucosamine moieties:

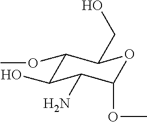

In one embodiment, the derivative has a molecular weight from 1000-10000 KDa, e.g. from 3000-6000 KDa, and it comprises from 1 to 1000, e.g. 10 to 50 primary amine functional groups.

In one embodiment, the oligomeric or polymeric saccharide comprises only glucosamine moieties. In a further embodiment, the saccharide is a chitosan oligomer or polymer. Chitosan is a natural, biodegradable linear polysaccharide comprising glucosamine units, which is used in water treatment, heavy metal removal, cosmetic additives, photographic papers, etc.¹¹ In another embodiment, the chitosan derivative is prepared from a low molecular weight chitosan oligosaccharide. In a further embodiment, the chitosan oligomer comprises up to 30 glycosamine moieties. In one example the chitosan derivative is prepared from chitosan oligosaccharide lactate, which is water-soluble, has a molecular weight of about 5000 and has about 25-30 primary amine functional groups.

In the context of the present invention, a derivative of an oligomeric or polymeric saccharide comprising glucosamine moieties is a molecule where a number of the amine groups on the glucosamine moieties are substituted by anchoring groups, e.g. chemical groups capable of chemisorbing to the surface of a nanoparticle, or groups capable of forming an interdigitated bilayer with a surfactant layer surrounding a nanoparticle. Examples of groups suitable for chemisorbing to the surface of a nanoparticle include thiol, amine, hydroxylamine, hydrazine, sulfide, sulfoxide, sulfone, phosphine, phosphite, phosphine oxide, carboxylate, thiocarboxylate, alcohol, carbene, imidazole, thiazole, or triazole groups, which groups are able to chemisorb to the surface of different types of nanoparticles. In one embodiment, the group suitable for chemisorbing to the surface of the nanoparticle is a thiol group and the nanoparticle comprises gold or silver. An example of a group suitable for forming an interdigitated bilayer with a surfactant layer surrounding the nanoparticle is an oleoyl group, which forms an interdigitated bilayer with cetyltrimethylammonium bromide (CTAB) coated nanoparticles.

In one embodiment, multiple anchoring groups can be introduced into the saccharide oligomer or polymer to bind the nanoparticle surface, which multiple anchoring points can improve the colloidal stability of the nanoparticle. For example, 1 to 1000, e.g. 10 to 25, of the amine groups in the glucosamine moieties can be substituted with the anchoring groups.

### Preparation of the Derivative

The primary amine groups of the glucosamine moieties can be substituted by the anchoring groups using standard chemical reactions that target primary amine groups. In one embodiment, the glucosamine-bearing oligomer or polymer can be reacted with iminothiolane hydrochloride to replace one or more of the amine groups with thiol groups. In another embodiment, the oligomer or polymer can be reacted with oleic anhydride to replace one or more of the amine groups with oleoyl groups. The amount of anchoring groups substituted onto the oligomer or polymer can be controlled by the molar amount of anchoring groups reacted with the glucosamine-bearing oligomer or polymer. For example, if about 7 molar equivalents of iminothiolane hydrochloride or oleic anhydride are used for each mole of chitosan oligomer, it can be expected that, assuming quantitative reactions, about 6 to 7 of the primary amine groups will be converted to thiol or oleoyl groups. The modification of chitosan can be confirmed and quantified by ¹H NMR.

### Functionalized Nanoparticles

In one embodiment, the nanoparticle has an average diameter of about 1 to 1000 nm, e.g. from 2 to 10 nm. The functionalized nanoparticles can take any shape, examples of which include nanospheres or nanorods. They can also vary in composition, and examples of suitable nanoparticles include noble metal nanoparticles, metal oxide nanoparticles (e.g. magnetic oxides), mixed oxide or mixed metal nanoparticles, polymeric or dendrimeric nanoparticles, hydroxyapatite nanoparticles, and quantum dots. Specific examples include gold nanoparticles, silver nanoparticles, ZnS—CdSe nanoparticles and iron oxide nanoparticles. In some embodiments, the nanoparticles comprise a surfactant layer on their surface.

The nanoparticles can be prepared according to known methods. For example, hydrophobic gold nanospheres can be synthesized by reducing a gold salt in toluene with tetrabutylammonium borohydride in the presence of long-chain fatty acid/ammonium salt. As another example, gold nanorods can be synthesized in an aqueous CTAB solution according to the published method.^{6a-c} After synthesis, the excess CTAB can be removed by ultracentrifugation, and the resulting nanorods, which are surrounded by a CTAB bilayer, can be redispersed in water.^{6d} The prepared nanoparticles are then coated by the anchoring group-bearing derivatives.

### Chemisorbtion of Chitosan Derivatives

In order to attach chitosan derivatives bearing anchoring groups that will chemisorb to the nanoparticle surface and displace surfactant molecules on the nanoparticle surface (e.g. derivatives bearing thiol groups), the nanospheres can be placed in an environment that permits reaction of the hydrophobic nanospheres with an aqueous solution comprising the derivative. For example, the nanoparticle can be dissolved in non-ionic reverse micelles, and then an aqueous solution of the derivative can be introduced. In one embodiment, the surfactant in the reverse micelle is selected to exhibit weaker interactions with the hydrophobic nanospheres so as to not disrupt the ligand exchange while preventing particle aggregation. The mixture can optionally be sonicated to facilitate reaction. Such a reaction proceeds by the exchange of surfactant molecules on the surface of the nanoparticle with the derivatives bearing the anchoring groups capable of chemisorbtion to the nanoparticle surface. The exchange of molecules can be partial or complete. The coated nanoparticles obtained can be isolated, e.g. by ethanol precipitation, and then dissolved in water. Chemisorbtion onto the nanoparticle surface allows both the hydrophobic nanoparticles and the water-soluble derivative to be solubilized. NMR studies can be used to confirm chemisorbtion onto the nanoparticle surface.

Use of derivatives bearing anchoring groups that will chemisorb to a nanoparticle surface is limited to nanoparticles where such a chemisorbtion will occur. For example, chitosan oligomers bearing thiol groups are suitable for coating gold or silver nanoparticles, as the interaction between the thiol groups is of sufficient strength to provide enhanced colloidal stability. As interaction of thiol groups with ZnS—CdSe and iron oxide nanoparticles is less, insoluble products are obtained.

Chemisorbed species are advantageous in that they afford a strong interaction between the nanoparticle and the coating.

### Interdigitated Chitosan Coating

For derivatives bearing anchoring groups that will form an interdigitated bilayer on the nanoparticle surface (e.g. chitosan oligomers bearing oleoyl groups), the inclusion of the derivative into the surfactant layer can be achieved by mixing a nanoparticle dispersion with a solution of the derivative. The mixture can optionally be sonicated to facilitate reaction. In such a reaction, the anchoring groups on the derivative can form an interdigitated bilayer with the surfactant layer (e.g. CTAB layer) present on the surface of the nanoparticle. The anchoring groups that form the interdigitated bilayer introduce multiple anchoring points within the surfactant layer on the nanoparticle and this provides a stable coating. NMR studies can be used to confirm formation of an interdigitated bilayer onto the nanoparticle surface.

This interdigitated bilayer coating method is beneficial in that it retains, at least in part, the original coating on the surface of the nanoparticle. This can be important in certain embodiments, such as in the case where the nanoparticle is a nanorod and the coating impacts the shape and colloidal stability of the nanorod. Further, this coating method does not require chemisorbtion of the chitosan derivative to the nanoparticle, which can be advantageous in those embodiments where there is no suitable anchoring groups to chemisorb to the nanoparticle surface or where the chemisorbtion achieved would be too weak to form a stable coating.

### Advantages and Opportunities for Further Functionalization

The coating obtained with the derivative as described herein is advantageous in that the presence of multiple attachment groups provides for enhanced stability.

Further, oligomeric and polymeric saccharides, such as chitosan, can be natural biomaterials that are biodegradable, biocompatible and water soluble, which properties makes these materials better choices in biological applications than the previously reported materials.

Chitosan-coated nanoparticles are water-soluble, colloidally stable, and robust against chemical conjugation steps.

Another attractive feature of the derivative-coated nanoparticles as described herein is the presence of surface primary amine groups, which groups can be used for bioconjugation with various molecules. Presence of the amine groups also permits the introduction of other functional groups, such as carboxy (e.g. for the formation of amide bonds), azido or acetylenic groups (e.g. for use in click chemistry), acrylate, ester, anhydride, amine, amide, and acetylene.

The chitosan-coated nanoparticles can also bear residual functional groups, such as thiol groups when a thiol-functionalized chitosan is chemisorbed to a nanoparticle and not all the thiol groups are chemisorbed to the nanoparticle surface.

Potential applications for such further functionalized nanoparticles include drug delivery, imaging, biosensing, targeting and tissue engineering. The obtained nanoparticles can be used directly in such applications, or they can be used as intermediates in the preparation of other molecular imaging agents for use in similar applications.

### EXAMPLES

The following examples are provided to illustrate the invention. It will be understood, however, that the specific details given in each example have been selected for purpose of illustration and are not to be construed as limiting the scope of the invention. Generally, the experiments were conducted under similar conditions unless noted.

### Example 1

### Chitosan Oligomer Modification

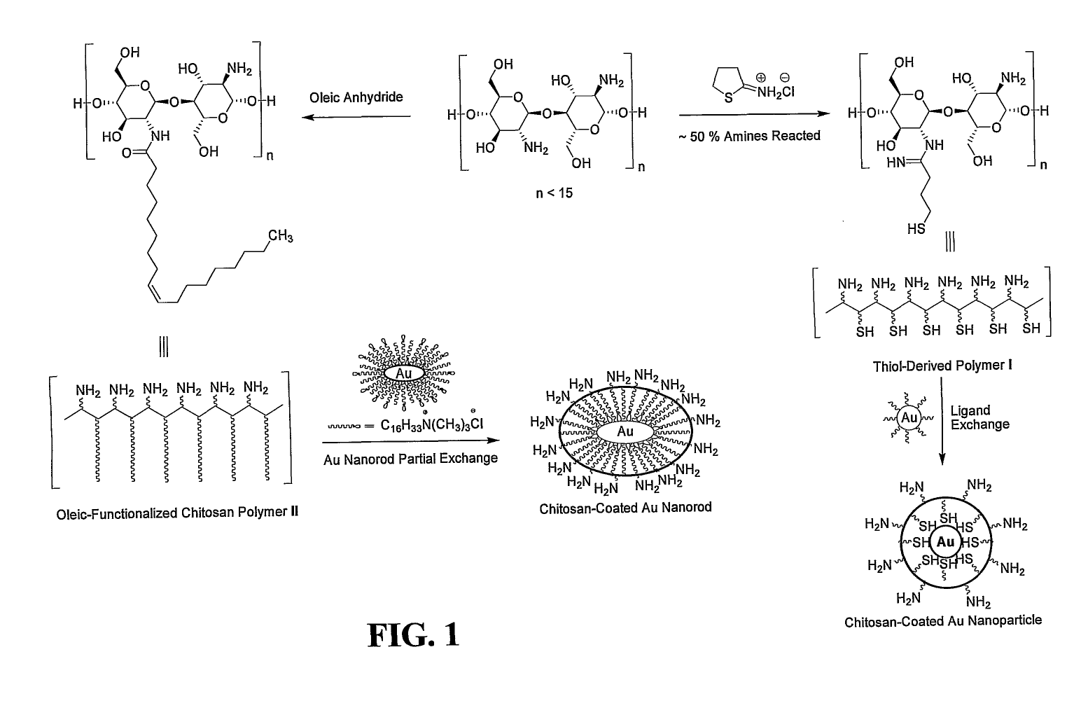

Chitosan modification pathways are illustrated in FIG. 1.

1a. Chitosan Oligosaccharide Modified with Iminothiolane Hydrochloride

An oven-dried, 10-ml reaction vial was charged with chitosan oligomer (1 g, 0.2 mmol) and phosphate buffer (5 mL) under argon atmosphere, and stirred until a clear homogeneous solution was obtained. A solution of iminothiolane hydrochloride (192 mg, 1.4 mmol) in phosphate buffer (pH 7.2, 1 mL) was added, and the mixture was stirred for 6 h at room temperature. The reaction mixture was concentrated under reduced pressure to a minimum volume, and the chitosan derivative was isolated by precipitation with methanol. The thiol-functionalized chitosan was purified by a repeated dissolution-precipitation method using water and methanol.¹H NMR analysis confirmed a quantitative incorporation of iminothiolane groups in the chitosan.

1b. Chitosan Oligosaccharide Modified with Oleic Anhydride

An oven-dried 10-ml reaction vial was charged with chitosan oligomer (1 g, 0.2 mmol), triethylamine (0.2 mL) and dry dimethylformamide (DMF) (5 mL) under argon atmosphere, and stirred until a clear homogeneous solution was obtained. Next, oleic anhydride (765 mg, 1.4 mmol) dissolved in dry DMF (1 mL) was added, and the mixture was stirred for 6 h at room temperature. The reaction mixture was concentrated under reduced pressure to a minimum volume of 1-2 mL, and the chitosan derivative was isolated by precipitation with methanol. The oleoyl-functionalized chitosan was purified by a repeated dissolution-precipitation method in DMF and methanol.¹H NMR analysis confirmed a quantitative incorporation of oleoyl groups in the chitosan.

### Example 2

### Coating of Hydrophobic Gold Nanospheres

Hydrophobic gold nanospheres of 3-4 nm were prepared in toluene in the presence of oleic acid and didodecyldimethyl ammonium bromide using a published procedure.^{2d} The Au concentration was about 10 mM. After synthesis, the samples were purified from free surfactants by ethanol precipitation. 1 mL of the solution was mixed with 500 μL of ethanol, and centrifuged at 16000 rpm for 5 min. The precipitated particles were dissolved in 2 mL of reverse micelles (0.5 mL of Igepal in 1.5 mL of cyclohexane). Next, an aqueous solution of the chitosan derivative from Example 1a (10 mg in 100 μL of water) was introduced and sonicated for 1 min. The particles were then precipitated by adding a few drops of ethanol. The precipitated particles were separated, washed with chloroform and ethanol, and then dissolved in water.

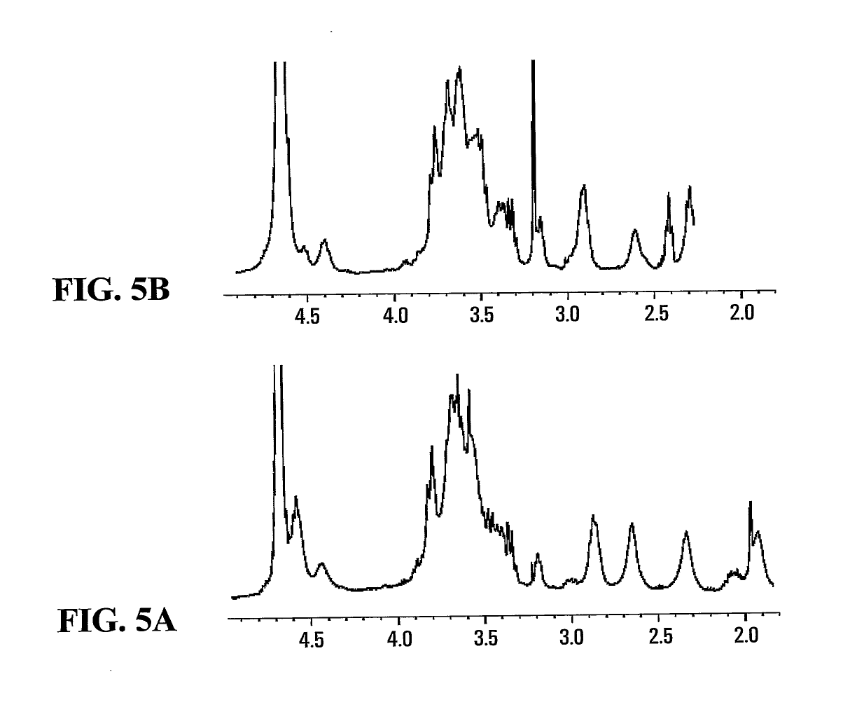

It could be seen by NMR that the original surfactant molecules were completely replaced by the chitosan derivative. The ¹H NMR spectra of the coated nanospheres (FIG. 5b) matches that of the modified chitosan (FIG. 5a), but the peaks are slightly shifted and broadened. This can be attributed to the strong interaction of the modified chitosan with the nanospheres.

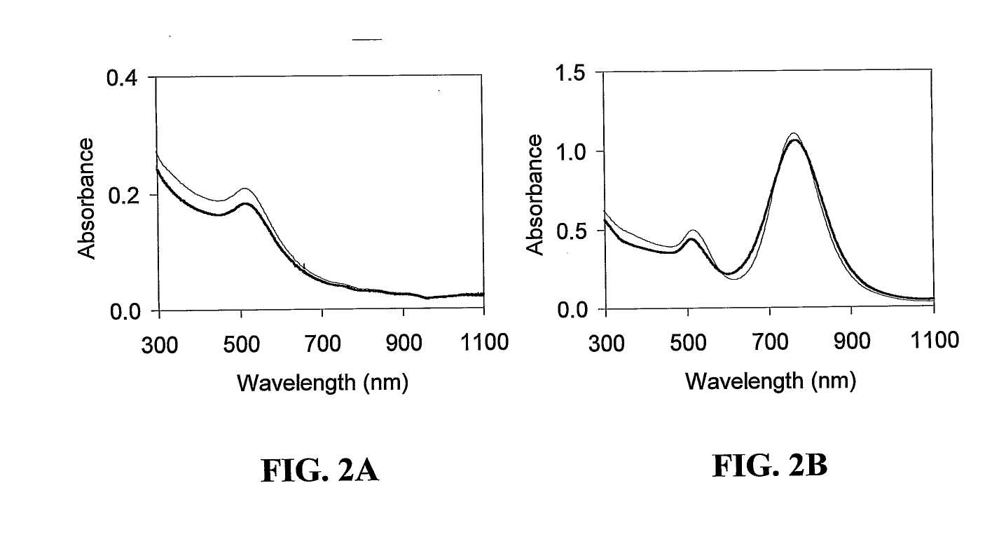

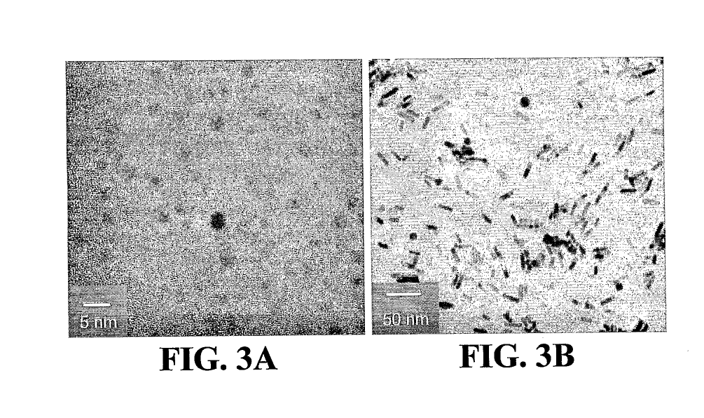

UV-visible spectroscopy and transmission electron microscopy (TEM) performed before and after the coating steps (FIGS. 2a and 3a) show that the particle size and shape remain unchanged upon coating. The coated nanospheres are also shown to be dispersed and non-aggregated.

### Example 3

### Coating of Gold Nanorods

The gold nanorods were synthesized in an aqueous CTAB solution using a published procedure.^{6a,c} The concentration of Au was about 1 mM, and excess CTAB was removed after the synthesis. 10.0 mL of the nanorod solution was centrifuged at 16000 rpm for 30 min. The precipitated particles were redissolved in 1.0 mL of water, and centrifuged again at 16000 rpm for 30 min. Finally, the particles were dissolved in 1.0 mL of water. 5 mg of the chitosan derivative from Example 1b was dispersed in 1.0 mL of water in another vial by 5 min of sonication, and mixed with the nanorod solution. The mixture was sonicated for 1 h. Next, insoluble chitosan was removed by centrifuging at 5000 rpm. Chitosan-coated nanorods were isolated by centrifugation, and then redispersed in water or aqueous buffer.

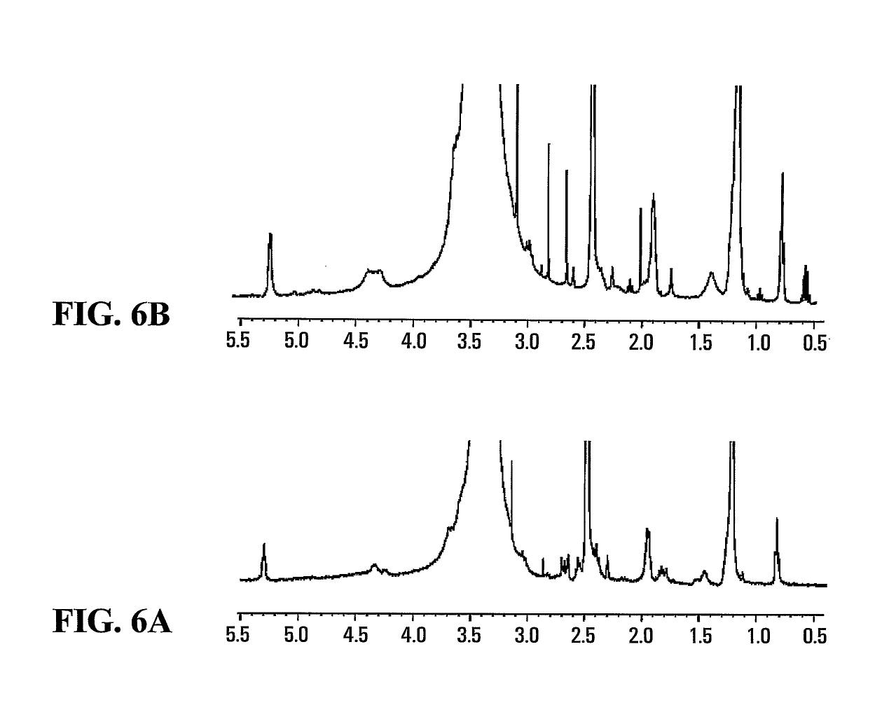

NMR studies of the chitosan-coated nanorods indicate that the CTAB was partially replaced by the chitosan derivative. The ¹H NMR spectra of the coated nanorods (FIG. 6b) matches that of the modified chitosan (FIG. 6a), but the peaks are slightly shifted and broadened. This can be attributed to the strong interaction of the modified chitosan with the nanorods. A possible structure is shown in FIG. 1.

UV-visible spectroscopy and transmission electron microscopy (TEM) performed before and after the coating steps (FIGS. 2b and 3b) show that the particle size and shape remain unchanged upon coating. The coated nanorods are also shown to be dispersed and non-aggregated.

Additional evidence that the chitosan derivative attached to the nanorod surface, and that CTAB was only partially replaced, was provided by the incorporation of more chitosan derivative from Example 1b (i.e. by repeating the chitosan introduction step), which led to a decrease in the water solubility of the nanorods. The nanorods were soluble in chloroform, however, where the chitosan derivative of Example 1b is soluble.

### Example 4

### Biotinylation of Gold Nanospheres and Nanorods

A chitosan-functionalized nanoparticle solution in borate buffer (pH 7.6) was mixed with a solution of N-hydroxy succinimide (NHS)-biotin (5 mg biotin dissolved in 200 μL of DMF), and incubated for 1 h. Next, free reagents were removed either by dialysis (for nanospheres) or by centrifugation (for nanorods). The biotinylated particles were then dissolved in tris buffer (pH 7.0).

Such binding of biotin to the nanoparticle can be used to confirm presence of the chitosan derivative on the nanoparticle surface as nanoparticles that do not have, absent the chitosan coating, the amine groups required for biotin functionalization.

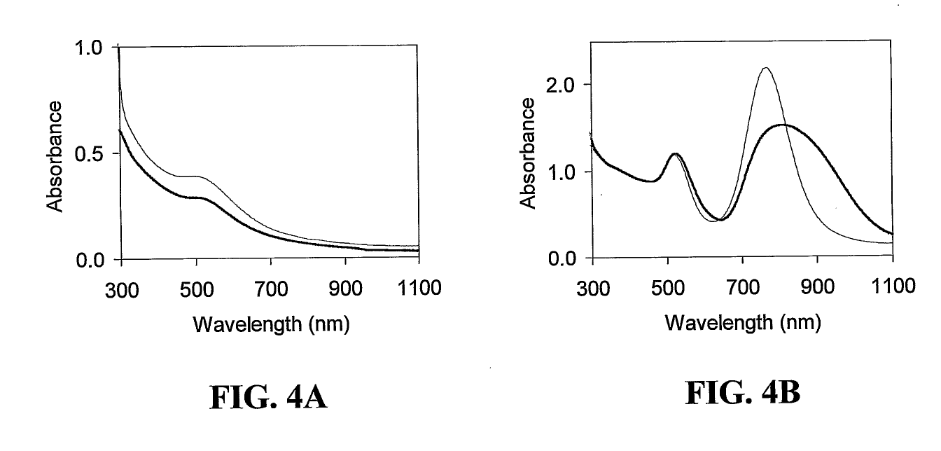

FIG. 4b shows the aggregation of biotinylated gold nanorods in the presence of streptavidin. Each streptavidin has four binding sites for biotin, and induces the aggregation of biotinylated nanoparticles. The nanorod aggregation is evident from the broadening and red-shifting of the surface plasmon band. It also leads to the precipitation of nanorods from solution. In comparison, FIG. 4a shows that nanospheres produced negligible shift in plasmon band, demonstrating an advantage of using anisotropic nanoparticles as sensors.

All publications, patents and patent applications cited in this specification are herein incorporated by reference as if each individual publication, patent or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

It must be noted that as used in this specification and the appended claims, the singular forms “a”, “an”, and “the” include plural reference unless the context clearly dictates otherwise. Unless defined otherwise all technical and scientific terms used herein have the same meaning as commonly understood to one of ordinary skill in the art to which this invention belongs.

### REFERENCES

1 (a) Eugenii, K.; Willner, I. Angew. Chem. Int. Ed. 2004, 43, 6042-6108. (b) Rosi, N L.; Mirkin, C. A. Chem. Rev. 2005, 105, 1547-1562. (c) Medintz, I. L.; Uyeda, H. T.; Goldman, E. R.; Mattoussi, H. Nature Mater. 2005, 4, 435-446.
2 (a) Murray, C. B.; Norris, D. J.; Bawendi, M. G. J. Am. Chem. Soc. 1993, 115, 8706-8715. (b) Li, J. J.; Wang, Y. A.; Guo, W.; Keay, J. C.; Mishima, T. D.; Johnson, M. B.; Peng, X. J. Am. Chem. Soc. 2003, 125, 12567-12575. (c) Brust, M.; Walker, M.; Bethell, D.; Schiffrin, D. J. J. Chem. Soc. Chem. Commun. 1994, 801-802. (d) Jana, N. R.; Peng, X. J. Am. Chem. Soc. 2003, 125, 14280-14281. (e) Jana, N. R.; Chen, Y.; Peng, X. Chem. Mater. 2004, 16, 3931-3935.
3 (a) Templeton, A. C.; Wuelfing, W. P.; Murray, R. W. Acc. Chem. Res. 2000, 33, 27-36. (b) Aldana, J.; Lavelle, N.; Wang, Y.; Peng, X. J. Am. Chem. Soc. 2005, 127, 2496-2504.
4 (a) Pellegrino, T.; Manna, L.; Kudera, S.; Liedl, T.; Koktysh, D.; Rogach, A. L.; Keller, S.; Radler, J.; Natile, G.; Parak, W. J. Nano Lett. 2004, 4, 703-707. (b) Yu. W.; Liu, H.; Liu, J.; Haley, K. N.; Treadway, J. A.; Larson, J. P.; Ge, N.; Paele, F.; Bruchez, M. P. Nature Biotech. 2003, 21, 41-46. (c) Zhelev, Z.; Ohba, H.; Bakalova, R. J. Am. Chem. Soc. 2006, 128, 6324-6325.
5 (a) Negishi, Y.; Tsukuda, T. J. Am. Chem. Soc. 2003, 125, 4046-4047. (b) Uyeda, H. T.; Medintz, I. L.; Jaiswal, J. K.; Simon, S. M.; Mattoussi, H. J. Am. Chem. Soc. 2005, 127, 3870-3878. (c) Wang, Z.; Tan, B.; Hussain, I.; Schaeffer, N.; Wyatt, M. F.; Brust, M.; Cooper, A. I. Langmuir 2007, 23, 885-895. (d) Guo, W.; Li, J. J.; Wang, Y. A.; Peng, X. J. Am. Chem. Soc. 2003, 125, 3901-3909. (e) Gao, X.; Cui, Y.; Levenson, R. M.; Chung, L. W. K.; Nie, S. Nat. Biotechnol. 2004, 22, 969-976.
6 (a) Murphy, C. J.; Jana, N. R. Adv. Mater. 2002, 14, 80-82. (b) Jana, N. R. Angew. Chem. Int. Ed. 2004, 43, 1536-1540. (c) Jana, N. R. Small, 2005, 1, 875-882. (d) Burda, C.; Chen, X.; Narayanan, R.; El-Sayed, M. A. Chem. Rev. 2005, 105, 1025-1102. (e) Mohamed, M. B.; Ismail, K. Z.; Link, S.; El-Sayed, M. A. J. Phys. Chem. B 1998, 102, 9370-9374. (f) Jana, N. R.; Gearheart, L.; Obare, S. 0.; Murphy, C. J. Langmuir 2002, 18, 922-927.
7 (a) Caswell, K. K.; Wilson, J. N.; Bunz, U. H. F.; Murphy, C. J. J. Am. Chem. Soc. 2003, 125, 13914-13915. (b) Liao, H.; Hafner, J. H. Chem. Mater. 2005, 17, 4636-4641. (c) Chen, C.-C.; Lin, Y.-P.; Wang, C.-W.; Tzeng, H.-C.; Wu, C.-H.; Chen, Y.-C.; Chen, C.-P.; Chen, L.-C.; Wu, Y.-C. J. Am. Chem. Soc. 2006, 128, 3709-3715. (d) Huff, T. B.; Hansen, M. N.; Zhao, Y.; Cheng, J.-X.; Wei, A. Langmuir 2007, 23, 1596-1599. (e) Pierrat, S.; Zins, I.; Breivogel, A.; Sonnichsen, C. Nano Lett. 2007, 7, 259-263. (f) Yu, C.; Irudayaraj, J. Anal. Chem. 2007, 79, 572-579.
8 (a) Obare, S. O.; Jana, N. R.; Murphy, C. J. Nano Lett. 2001, 1, 601-603. (b) Pastoriza-Santos, I.; Perez-Juste, J.; Liz-Marzan, L. M. Chem. Mater. 2006, 18, 2465-2467. (c) Wang, C.; Ma, Z.; Wang, T.; Su, Z. Adv. Funct. Mater. 2006, 16, 1673-1678.
9 Takahashi, H.; Niidome, Y.; Niidome, T.; Kaneko, K.; Kawasaki, H.; Yamada, S. Langmuir 2006, 22, 2-5. Gole, A.; Murphy, C. J. Chem. Mater. 2005, 17, 1325-1330.
11 (a) Qiu, X.; Leporatti, S.; Donath, E.; Mohwald, H. Langmuir 2001, 17, 5375-5380. (b) Boddu, V. M.; Abburi, K.; Talbott, J. L.; Smith, E. D. Environ. Sci. Technol. 2003, 37, 4449-4456. (c) Zhang, H.; Oh, M.; Allen, C.; Kumacheva, E. Biomacromolecules 2004, 5, 2461-2468. (d) Lin, Y.-H.; Mi, F.-L.; Chen, C.-T.; Chang, W.-C.; Peng, S.-F.; Liang, H.-F.; Sung, H.-W. Biomacromolecules 2007, 8, 146-152.

1. A derivative of a low molecular weight oligomeric saccharide comprising glucosamine moieties having primary amine groups and optionally non-primary amine groups, wherein the number of primary amine groups exceed the number of non-primary amine groups when present, and wherein one or more primary amine groups are substituted by anchoring groups that chemisorb to the surface of a nanoparticle or form an interdigitated bilayer with a surfactant layer surrounding the nanoparticle.

2. The derivative according to claim 1, wherein the oligomeric saccharide is an oligo-glucosamine.

3. The derivative according to claim 1, wherein the oligomeric saccharide is a chitosan oligomer.

4. The derivative according to claim 1, wherein the anchoring group is a thiol, amine, hydroxylamine, hydrazine, sulfide, sulfoxide, sulfone, phosphine, phosphite, phosphine oxide, carboxylate, thiocarboxylate, alcohol, carbene, imidazole, thiazole, triazole, or oleoyl group.

5. The derivative according to claim 1, wherein the anchoring group is a thiol group.

6. The derivative according to claim 1, wherein the anchoring group is an oleoyl group.

7. The derivative according to claim 1, which has a molecular weight of from about 1000 to about 10000 Da.

8. The derivative according to claim 1, which has a molecular weight of from about 3000 to about 6000 Da.

9. The derivative according to claim 1, which comprises 1 to 1000 anchoring groups.

10. The derivative according to claim 1, which comprises from 10 to 25 anchoring groups.

11. The derivative according to claim 1, which comprises 1 to 1000 primary amine groups.

12. The derivative according to claim 1, which comprises from 10 to 50 primary amine groups.

13. (canceled)

14. The derivative according to claim 1, which has a molecular weight of about 5000 Da, 6 or 7 anchoring groups, and 18 to 24 primary amine groups.

15. A functionalized nanoparticle, comprising:
a nanoparticle; and
a derivative of a low molecular weight oligomeric saccharide comprising glucosamine moieties having primary amine groups and optionally non-primary amine groups, wherein the number of primary amine groups exceed the number of non-primary amine groups when present, and wherein one or more primary amine groups are substituted by anchoring groups that chemisorb to the surface of the nanoparticle or form an interdigitated bilayer with a surfactant layer surrounding the nanoparticle.

16. The functionalized nanoparticle according to claim 15, wherein the nanoparticle is a noble metal nanoparticle, metal oxide nanoparticle, mixed oxide or mixed metal nanoparticle, polymeric or dendrimeric nanoparticle, hydroxyapatite nanoparticle, or quantum dot.

17. The functionalized nanoparticle according to claim 15, wherein the nanoparticle is a gold, silver, ZnS—CdSe or iron oxide nanoparticle.

18. The functionalized nanoparticle according to claim 15, wherein the nanoparticle is a nanosphere or of a nanorod.

19. The functionalized nanoparticle according to claim 15, which is a gold nanosphere, a silver nanosphere, a ZnS—CdSe nanosphere, an iron oxide nanosphere or a gold nanorod.

20-24. (canceled)

25. A method for forming a functionalized nanoparticle as defined in claim 15, comprising:
reacting a derivative, wherein the derivative comprises a low molecular weight oligomeric saccharide comprising glucosamine moieties having primary amine groups and optionally non-primary amine groups, wherein the number of primary amine groups exceed the number of non-primary amine groups when present, and wherein one or more primary amine groups are substituted by anchoring groups that chemisorb to the surface of a nanoparticle or form an interdigitated bilayer with a surfactant layer surrounding the nanoparticle, with a nanoparticle.

26-34. (canceled)

35. A method of treating or diagnosing a patient in need thereof, comprising: administering a functionalized nanoparticle as defined in claim 15 as a molecular imaging agent, a biosensing agent or a drug delivery agent.
